(19) United States
(12) Patent Application Publication
(10) Pub. No.: US 2015/0316497 A1
(43) Pub. Date: Nov. 5, 2015

(54) WIRELESS SUBSOIL TENSION SENSOR

(71) Applicant: Cheng-Hung CHANG, Hsinchu City (TW)

(72) Inventor: Cheng-Hung CHANG, Hsinchu City (TW)

(21) Appl. No.: 14/266,309

(22) Filed: Apr. 30, 2014

(51) Int. Cl. G01N 27/12; G01N 33/24

(52) U.S. Cl. CPC G01N 27/121; G01N 33/246

(57) ABSTRACT

A wireless subsoil tension meter is disclosed, including an upper housing part, a middle housing part, a lower housing part, and a sensor module. The upper, middle and lower housing parts are assembled to form a sealed space to house a sensor module and liquid. The upper housing part has a tubular body shape. The middle housing part has a funnel body shape, with a larger top and the smaller bottom, the top end of the middle housing part is slightly smaller than the inside the bottom end of the outer tube of the upper housing part for easy assembly and tight fit. The lower housing part is has an elongated dome shape. When assembled, the sensor module is housed inside the assembly, which can further be applied to an extendable wireless soil measurement apparatus.

### FIELD OF THE INVENTION

The present invention generally relates to a wireless subsoil tension sensor.

### BACKGROUND OF THE INVENTION

Real-time subsoil tension is by far the most effective way to manage agricultural produce irrigation through monitoring the moisture level and water availability in the soil so as to reduce water waste without stressing the agricultural crops.

A conventional tension meter is made into a sealed tube forming a chamber entirely filled with liquid and a porous tip connected to one end of the tube. The porous tip is buried under soil. The moisture in the soil surrounding the porous tip and the liquid inside the chamber forming liquid contact. The relatively dry soil will slowly draw liquid from the tube through the porous tip. By measuring the remaining liquid inside the tube, the moisture contents in the soil can be determined.

FIG. 1 shows a schematic view of the structure a conventional wireless subsoil tension meter. As shown in FIG. 1, a conventional wireless tension meter has a body 100 which includes a tubular housing 110 with a lower end 111 and an upper end 112, a porous material tip 120, a head 130, and an antenna 140. The porous material tip 120 is mounted to the lower end 111 of the tubular housing 110. The porous material tip 120 has a first section which extends in the tubular housing 110 and a second section which is in direct contact with the porous medium when inserted therein. The head 130 is mounted to the upper end 112 of the tubular housing 110. The antenna 140 is mounted to the head 130. The head 130 and the antenna 140 extend above the porous medium when the tension meter is inserted therein. The tubular housing 110 has a peripheral wall which defines a fluid chamber therein. The fluid chamber extends from the first end 111 to the second end 112 of the tubular housing 110.

### SUMMARY OF THE INVENTION

The present invention has been made to overcome the above-mentioned drawback of conventional wireless subsoil tension measurement system. The primary object of the present invention is to provide a wireless subsoil tension sensor that provides deployment flexibility and ease.

An exemplary embodiment of the present invention discloses a wireless subsoil tension meter, including an upper housing part, a middle housing and a lower housing part, assembled to form a sealed space to house a sensor module and liquid; wherein the upper housing part having a tubular body shape and further including an outer tube, an inner tube and a ring-shaped plate connecting the outer tube and the inner tube; the inner tube further including a bottom with an opening, and a protruding wall surrounding the opening; when the sensor module being disposed inside the inner tube, the sensor module sitting the protruding wall to seal the opening; the ring-shaped plate connecting the outer tube and the inner tube being disposed with a small hole; the upper housing part further including a ring-shaped sealing piece with a plug to seal the small hole on the ring-shape plate, the ring-shaped sealing piece being disposed on the ring-shaped plate; the middle housing part having a funnel body shape, with a larger top and the smaller bottom, the top end of the middle housing part being slightly smaller than the inside the bottom end of the outer tube of the upper housing part, the middle housing part further including a bottom ring and a top protruding wall; the top protruding wall having a tubular shape and being an extension from the top end of the funnel part of the middle housing part; the top protruding wall is slightly smaller in size to fit tightly inside the outer wall of the upper housing part; the bottom ring being connected to the bottom end of the funnel part of the middle housing part ; the lower housing part further including a top ring, connected to the top end of the lower housing part.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

The present invention can be understood in more detail by reading the subsequent detailed description in conjunction with the examples and references made to the accompanying drawings, wherein:

FIG. 1 shows a schematic view of the structure a conventional wireless subsoil tension meter;

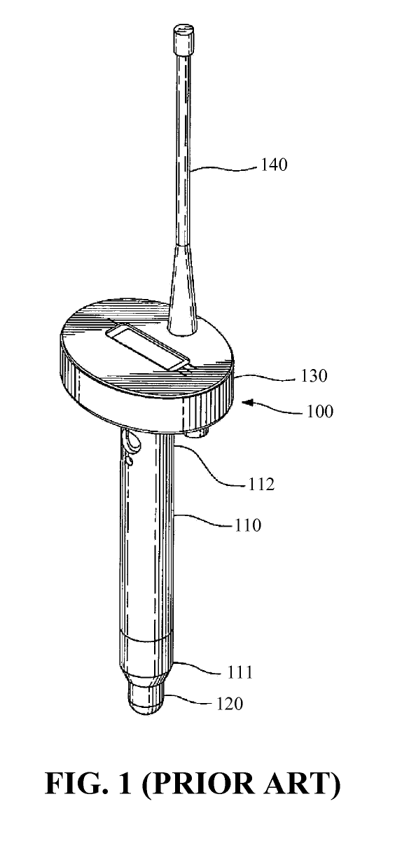

FIG. 2 shows a schematic cross-sectional view of a wireless subsoil tension meter according to an embodiment of the present invention; and

FIG. 3 shows a schematic cross-sectional view of wireless subsoil tension sensor of the present invention applied to an extendable wireless soil measurement apparatus; and

FIG. 4 shows a schematic cross-sectional view of the of wireless subsoil tension sensor of the present invention applied to another embodiment of an extendable wireless soil measurement apparatus

### DETAILED DESCRIPTION OF THE PREFERRED EMBODIMENTS

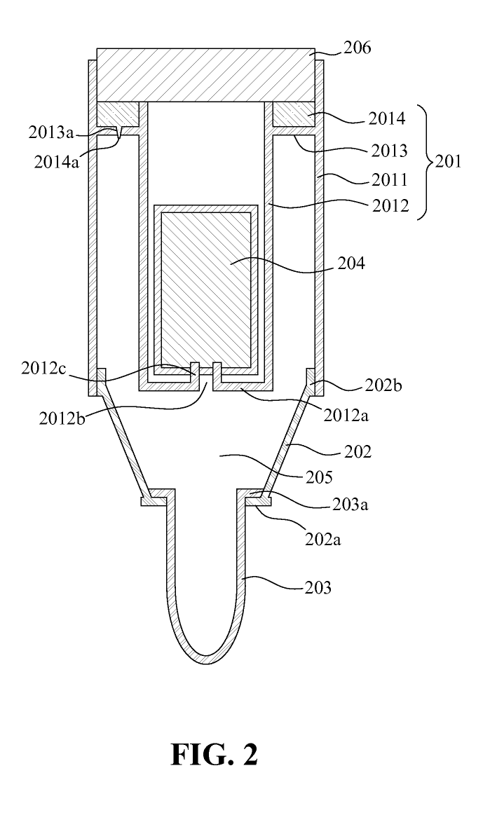

FIG. 2 shows a schematic cross-sectional view of a wireless subsoil tension meter according to an embodiment of the present invention. As shown in FIG. 2, a wireless subsoil tension meter including an upper housing part 201, a middle housing part 202, a lower housing part 203, and a sensor module 204. The upper housing part 201, the middle housing part 202, and the lower housing part 203 are assembled to form a sealed space 205 to house a sensor module 204 and liquid. The upper housing part 201 has a tubular body shape, the middle housing part 202 has a funnel shape and the lower housing part 203 has an elongated dome shape. In the instant embodiment, the top end of the middle housing part 202 is slightly reduced for easy assembly and tight fit inside the bottom end of the upper housing part 201, and the top end of the lower housing part 203 is slightly reduced for easy assembly and tight fit inside the bottom end of the middle housing part 203. As such, the upper housing part 201, the middle housing part 202 and the lower housing part 203 can form a tightly sealed space 205 for storing liquid.

The upper housing part 201 further includes an outer tube 2011, an inner tube 2012 and a ring-shaped plate 2013 connecting the outer tube 2011 and the inner tube 2012. The inner tube 2012 further includes a bottom 2012a with an opening 2012b, and a protruding wall 2012c surrounding the opening 2012b. The protruding wall 2012c has a short height so that when the sensor module 204 is disposed inside the inner tube 2012, the sensor module 204 sits the protruding wall 2012c to seal the opening 2012b. The ring-shaped plate 2013 connecting the outer tube 2011 and the inner tube 2012 is disposed with a small hole 2013a. The upper housing part 201 further includes a ring-shaped sealing piece 2014 with a plug 2014a to seal the small hole 2013a on the ring-shape plate 2013. The ring-shaped sealing piece 2014 is disposed on the ring-shaped plate 2013. The outer tube 2011, the inner tube 2012 and the ring-shaped plate 2013 are monolithically manufactured, for example, with plastic. The ring-shaped sealing piece 2014 may be, for example, made of rubber. When the ring-shaped sealing piece 2014 is assembled onto the ring-shaped plate 2013, the height of the ring-shaped sealing piece 2014 is at the same level as the top end of the inner tube 2012, and both are slightly lower than the top end of the outer tube 2011. As such, an upward-facing concave is formed inside the outer tube 2011 and above the inner tube 2012.

As aforementioned, the middle housing part 202 has a funnel body shape, with a larger top and the smaller bottom. In the instant embodiment, the top end of the middle housing part 202 is slightly smaller than the inside the bottom end of the outer tube 2011 of the upper housing part 201 for easy assembly and tight fit. In the instant embodiment, the middle housing part 202 further comprises a bottom ring 202a and a top protruding wall 202b. The top protruding wall 202b has a tubular shape and is an extension from the top end of the funnel part of the middle housing part 202. The top protruding wall is slightly smaller in size to fit tightly inside the outer wall 2011 of the upper housing part 201. The bottom ring 202a serves as a bottom of the middle housing part 202 and is connected to the bottom end of the funnel part of the middle housing part 202. The center hole of the bottom ring 202a is for the insertion of the lower housing pat 203 when assembled. In the present embodiment, the middle housing part 202 may be made of plastic.

The lower housing part 203 further includes a top ring 203a, connected to the top end of the lower housing part 203. The top ring 203a of the lower housing part 203 matches the bottom ring 202a of the middle housing part 202. The lower housing part 203 is made of porous ceramic to allow moisture osmosis.

To assembly, the lower housing part 203 is first inserted into the middle housing part 202, with the elongated dome of the lower housing part 203 passing through the center hole of the bottom ring 202a of the middle housing part until the top ring 203a of the lower housing part 203 sits on the bottom ring 202a of the middle housing part 202. Then, the top protruding all 202b of the middle housing part is inserted into the bottom end of the outer tube 2011 of the upper housing part 201. Glue may be applied to enhance the assembly at the engaged portion between the three housing parts. The sensor module 204 is placed into the inner tube 2012, and the liquid is injected into the sealed space 205 through the small hole 2013a on the ring-shape plate 2013. Then, the ring-shaped sealing piece 2014 is disposed on top of the ring-shape plate 2013 with the plug 2014a plugged into the small hole 2013a to complete the sealing of the liquid contained inside the sealed space 205.

It should be noted that the sensor module 204 further includes a pressure sensor unit and a temperature sensor unit for detecting soil tension, a transceiver for wireless communication and a power supply unit, such as, a button cell battery. Additional sensor units, such as, temperature sensor unit, may also be included.

As shown in FIG. 2, the wireless subsoil tension meter further includes a cap element 206, disposed inside the upward-facing concave formed inside the outer tube 2011 and above the inner tube 2012. In the instant embodiment, the cap element 206 may be, for example, a plastic screw.

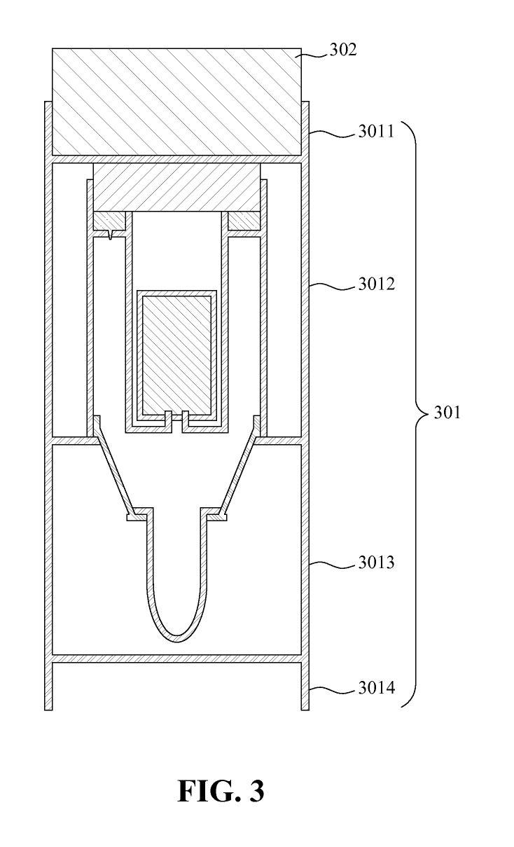

FIG. 3 shows a schematic view of wireless subsoil tension sensor of the present invention applied to an extendable wireless soil measurement apparatus. As shown in FIG. 3, the wireless subsoil tension sensor of the present invention may be housed inside a sensor housing 301, which further includes a first segment 3011, a second segment 3012, a third segment 3013 and a fourth segment 3014, wherein the four segments may be connected together to form an integrated shell of a cylindered shape. The sensor housing 301 is preferably made of metal, porous ceramic or plastic material. The first segment 3011 is structured to include a cylindered wall and a bottom to form a dish. The second segment 3012 is also structured to include a cylindered wall and a bottom. When connected, the bottom of the first segment 3011 and the second segment 3012 form a cavity for housing the upper housing part 201 of the subsoil tension sensor of the present invention to seal and protect from contacting the soil. The bottom has an opening for allowing the middle housing part 202 and lower housing part 203 of the subsoil tension sensor to extend into the cavity formed by the third segment 3013, which has a structure similar to the first segment 3011. When connected to the second segment 3012, a cavity is formed for housing the middle housing part 202 and the lower housing part 203 of the subsoil tension sensor of the present invention. The fourth segment 3014 is structured as a cylindered wall attached to the bottom of the third segment 3013, and may be considered as an extension of the third segment 3013. When two sensors are assembled, the fourth segment 3014 of the previous sensor and the first segment 3011 of the next sensor are engaged by an engaging element 302. As such, the sensors can be connected head-to-tail through engaging elements 302 to form a pole.

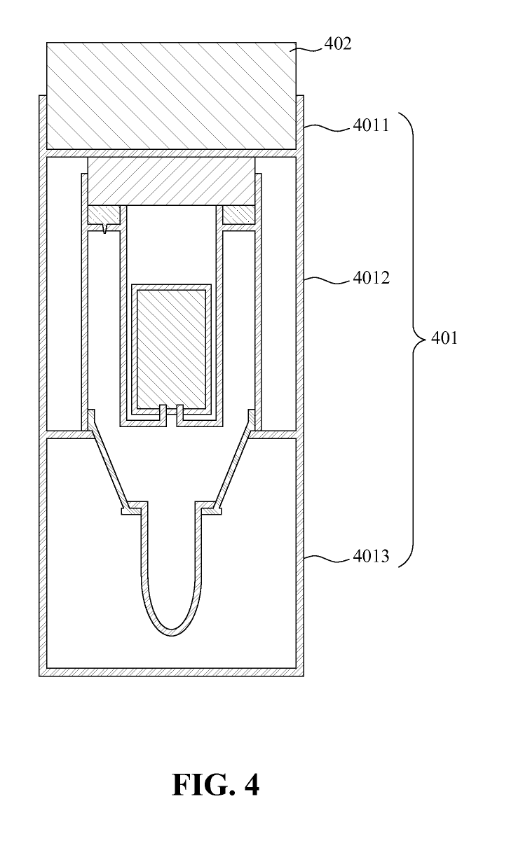

FIG. 4 shows a schematic view of the of wireless subsoil tension sensor of the present invention applied to another embodiment of an extendable wireless soil measurement apparatus. As shown in FIG. 4, the sensor housing 401 includes a first segment 4011, a second segment 4012 and a third segment 4013. This embodiment can be used as a terminal of the pole.

In summary, the structure of the wireless subsoil tension meter of the present invention allows a plurality of wireless subsoil tension meters to form the probe so as to provide ease and flexibility of the deployment of the meters to accommodate the various underground conditions.

An exemplary embodiment of the present invention discloses a wireless subsoil tension meter, including an upper housing part, a middle housing and a lower housing part, assembled to form a sealed space to house a sensor module and liquid; wherein the upper housing part having a tubular body shape and further including an outer tube, an inner tube and a ring-shaped plate connecting the outer tube and the inner tube; the inner tube further including a bottom with an opening, and a protruding wall surrounding the opening; when the sensor module being disposed inside the inner tube, the sensor module sitting the protruding wall to seal the opening; the ring-shaped plate connecting the outer tube and the inner tube being disposed with a small hole; the upper housing part further including a ring-shaped sealing piece with a plug to seal the small hole on the ring-shape plate, the ring-shaped sealing piece being disposed on the ring-shaped plate; the middle housing part having a funnel body shape, with a larger top and the smaller bottom, the top end of the middle housing part being slightly smaller than the inside the bottom end of the outer tube of the upper housing part, the middle housing part further including a bottom ring and a top protruding wall; the top protruding wall having a tubular shape and being an extension from the top end of the funnel part of the middle housing part; the top protruding wall is slightly smaller in size to fit tightly inside the outer wall of the upper housing part; the bottom ring being connected to the bottom end of the funnel part of the middle housing part ; the lower housing part further including a top ring, connected to the top end of the lower housing part.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A wireless subsoil tension meter, comprising:
an upper housing part, having a tubular body shape, and further comprising an outer tube, an inner tube and a ring-shaped plate connecting the outer tube and the inner tube; the inner tube further comprising a bottom with an opening, and a protruding wall surrounding the opening; when the sensor module being disposed inside the inner tube, the sensor module sitting the protruding wall to seal the opening; the ring-shaped plate connecting the outer tube and the inner tube being disposed with a small hole; the upper housing part further comprising a ring-shaped sealing piece with a plug to seal the small hole on the ring-shape plate, the ring-shaped sealing piece being disposed on the ring-shaped plate;
a middle housing part, having a funnel shape, with a larger top end and a smaller bottom end, and further comprising a top protruding wall connected to the top end of the funnel shape of the middle housing part and a bottom ring connected to the bottom end of the funnel shape of the middle housing part;
a lower housing part, having an elongated dome shape, further comprising a top ring connected to the top end of the elongated dome shape of the lower housing part;
matching the tubular body shape of the upper housing part, top end of the lower housing part being slightly reduced to fit tightly inside bottom end of the upper housing part to form a tightly sealed space for storing liquid; and
a sensor module, for sensing soil tension and transmitting soil condition through a wireless means;
wherein the top protruding wall of the middle housing part being slightly smaller than the bottom end of the outer tube of the upper housing part, and the top ring of the lower housing part matching the bottom ring of the middle housing part; when assembled, the sensor module sitting inside the inner tube of the upper housing part, and the top ring of the power housing part sitting on the bottom ring of the middle housing part.

2. The wireless subsoil tension meter as claimed in claim 1, when the ring-shaped sealing piece is assembled onto the ring-shaped plate, the height of the ring-shaped sealing piece is at the same level as top end of the inner tube, and both are lower than top end of the outer tube to form an upward-facing concave inside the outer tube and above the inner tube.

3. The wireless subsoil tension meter as claimed in claim 1, wherein the outer tube, the inner tube and the ring-shape plate of the upper housing part are monolithically manufactured with plastic.

4. The wireless subsoil tension meter as claimed in claim 1, wherein the lower housing part is made of porous ceramic.

5. The wireless subsoil tension meter as claimed in claim 1, wherein the sensor module further comprises a pressure sensor unit and a temperature sensor unit for detecting soil tension, a transceiver for wireless communication and a power supply unit, and a power supply unit.
